(19) United States
(12) Patent Application Publication
(10) Pub. No.: US 2002/0040190 A1
(43) Pub. Date: Apr. 4, 2002

(54) Surgical microscope arrangement

(76) Inventors: Ulrich Nagele, Bartholoma (DE); Martin Pelzer, Konigsbronn (DE); Christian Duschek, Horn (DE); Ulrich Gold, Aalen (DE); Erik Duver, Oberkochen (DE); Dieter Quendt, Essingen (DE)

Correspondence Address: Walter Ottesen, Patent Attorney, P.O. Box 4026, Gaithersburg, MD, 20885-4026, US

(21) Appl. No.: 09/964,429

(22) Filed: Sep. 28, 2001

(30) Foreign Application Priority Data
Sep. 30, 2000 (DE) 100 48 546.4

(51) Int. Cl. A61B 6/00

(52) U.S. Cl. 600/476; 606/130

(57) ABSTRACT

A surgical microscope (1) has an objective (12) for viewing an object (2) and a device for detecting the spatial position of points of the object. The surgical microscope (1) includes a first image sensor (10) and a second image sensor (11) which are arranged at a spacing from each other.

### FIELD OF THE INVENTION

The invention relates to a surgical microscope arrangement including a surgical microscope having an objective for viewing an object and having a device for detecting the spatial position of points of the object.

### BACKGROUND OF THE INVENTION

A surgical microscope arrangement of the above type is disclosed, for example, in U.S. Pat. No. 5,999,837. In this surgical microscope, the position of an object in the field of view of the surgical microscope relative to the microscope can be determined by detecting the position of the surgical microscope relative to the operating room and by detecting the position of the corresponding object relative to the operating room, for example, with the aid of a pointer.

### SUMMARY OF THE INVENTION

It is an object of the invention to provide a surgical microscope arrangement with which the detection of spatial position can be carried out easily with greater accuracy while being less susceptible to disturbances.

The surgical microscope arrangement of the invention includes: a surgical microscope for viewing an object; the surgical microscope having an objective defining an optical axis; a device for detecting the spatial position of points on the object; and, the device including first and second image sensor units mounted on the surgical microscope so as to be mutually separated at a predetermined distance.

The image sensors of the surgical microscope are arranged at a spacing to each other. With these image sensors, the detection of spatial position can take place directly from the stereo image pairs, which are detected by the two image sensors, via an evaluation method disclosed, for example, in German patent publication 3,720,019. In this way, the spatial position relative to the surgical microscope can be detected with a lesser number of measurements as was required previously.

The problem of shading associated with the arrangement of U.S. Pat. No. 5,999,837 between the image sensors and the object is considerably reduced with the surgical microscope according to the invention.

### BRIEF DESCRIPTION OF THE DRAWINGS

The invention will now be described with reference to the drawings wherein:

FIG. 1 is a schematic showing the surgical microscope arrangement of the invention in use together with a surgical instrument; and,

FIG. 2 is a perspective view of the surgical microscope of the surgical microscope arrangement of the invention.

### DESCRIPTION OF THE PREFERRED EMBODIMENTS OF THE INVENTION

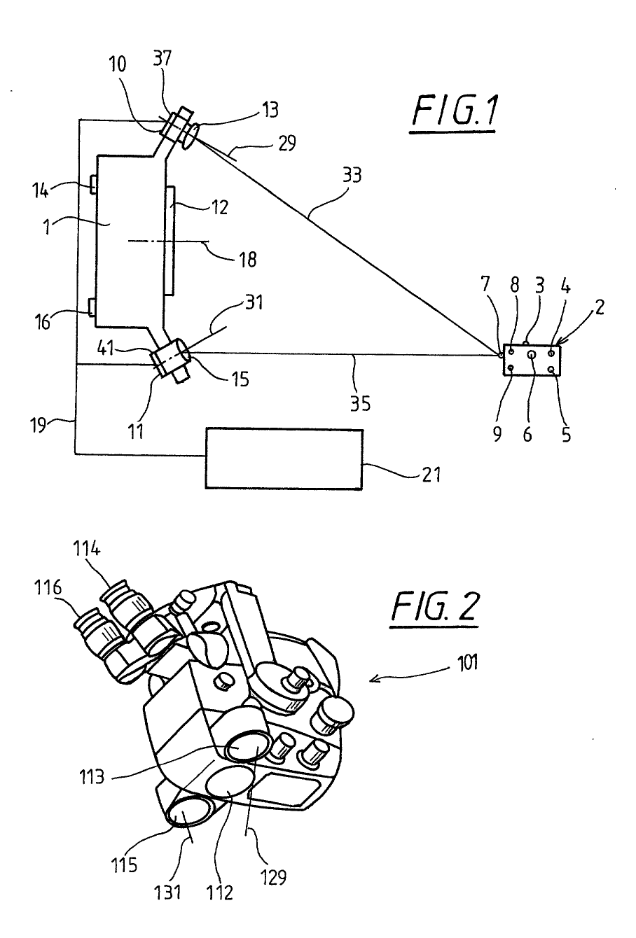

FIG. 1 shows a surgical microscope 1 according to the invention. A surgical instrument 2 is disposed in the field of view of the surgical microscope 1 and includes markers 3 to 9 configured as spherical reflectors.

The surgical microscope 1 includes an objective 12, oculars 14 and 16 as well as image sensors 10 and 11 arranged next to the objective 12. The image sensors 10 and 11 can, for example, be configured as CCD arrays. The two image sensors 10 and 11 are arranged at a spacing from each other and with different alignment to each other for generating stereo image pairs which can be evaluated with respect to coordinates.

The surgical microscope 1 furthermore includes imaging devices 13 and 15 configured as camera objectives. The imaging devices 13 and 15 image the objects in the field of view of the surgical microscope 1 and, for example, the markers 3 to 9 of the surgical instrument onto the image sensors 10 and 11, respectively. Reference numeral 29 identifies the optical axis of image sensor 10 and reference numeral 31 identifies the optical axis of image sensor 11. Reference numeral 33 identifies the chief ray of the beam contributing to the imaging of the marker 7 on the image sensor 10 and reference numeral 35 identifies the chief ray of the beam contributing to the imaging of the marker 7 on image sensor 11.

The images detected by the image sensors 10 and 11 are transmitted via a signal line 19 to an evaluation unit 21. From these images, the evaluation unit 21 determines the position coordinates of the markers 3 to 9 in a coordinate system fixed by the arrangement of the image sensors 10 and 11 and referred thereby to the surgical microscope 1.

The surgical microscope of the invention can also be equipped with three or more image sensors. Then, in lieu of an image pair, an image triple, et cetera, would evaluated by the evaluation unit 21.

FIG. 2 shows the surgical microscope according to the invention in a perspective view. Elements corresponding to the elements of the surgical microscope 1 of FIG. 1 have the same reference numeral increased by a factor of 100 in FIG. 2. Reference is made to FIG. 1 for the description of these elements.

In FIG. 2, it can be seen that the imaging devices 113 and 115 are arranged laterally to the object 112 and accommodate the object 112 therebetween. For relatively low outer dimensions of the surgical microscope, this ensures a relatively large and therefore favorable stereo basis for the image evaluation of the stereo image pair.

It is understood that the foregoing description is that of the preferred embodiments of the invention and that various changes and modifications may be made thereto without departing from the spirit and scope of the invention as defined in the appended claims.

What is claimed is:

1. A surgical microscope arrangement comprising:
a surgical microscope for viewing an object;
said surgical microscope having an objective defining an optical axis;
a device for detecting the spatial position of points on said object; and,
said device including first and second image sensor units mounted on said surgical microscope so as to be mutually separated at a predetermined distance.

2. The surgical microscope arrangement of claim 1, said first image sensor unit including a first image sensor and a first imaging device for imaging said object onto said first image sensor; said second image sensor unit including a second image sensor and a second imaging device for imaging said object onto said second image sensor; and, said first and second imaging devices defining first and second imaging axes inclined with respect to said optical axis of said objective.

3. The surgical microscope arrangement of claim 2, wherein said first and second imaging devices are disposed at respective locations to the right and to the left of said objective.

4. The surgical microscope arrangement of claim 3, further comprising an evaluation unit connected to said first and second imaging sensors; and, said evaluation unit functioning to determine the spatial position of a point on said object relative to said surgical microscope from the following: the location of said point in the image detected by said first image sensor; the location of said point in the image detected by said second image sensor; and, the spatial disposition of said first and second image sensors and the first and second imaging devices.
